(19) United States
(12) Patent Application Publication
(10) Pub. No.: US 2005/0049415 A1
(43) Pub. Date: Mar. 3, 2005

(54) Process for preparing enantiomerically pure alpha phenyl-alpha (6,7-dihydro-4h-thieno[3,2-c]pyridin-5-yl)-acetic acid derivatives

(76) Inventors: Ramakrishna Arul, Taloja (IN); Rawat Ajaysingh, Taloja (IN); Mahesh Gadakar, Taloja (IN); Abhinay Pise, Taloja (IN); Rajesh Rao, Taloja (IN); Jayaraman Venkat Raman, Taloja (IN)

Correspondence Address: JONES DAY, 222 EAST 41ST ST, NEW YORK, NY, 10017, US

(21) Appl. No.: 10/493,994

(22) PCT Filed: Oct. 28, 2002
(86) PCT No.: PCT/GB02/04856

(30) Foreign Application Priority Data
Oct. 26, 2001 (GB) 01257088

(51) Int. Cl. C07D 498/02

(52) U.S. Cl. 546/114

(57) ABSTRACT

A process for preparing a substantially enantiomerically pure compound of formula (IV), or a pharmaceutically acceptable salt thereof wherein: R is hydrogen or a Cl alkyl group and X, Y and Z are any atom or group, comprising a step of isolating a substantially enantiomerically pure compound of formula (V) wherein: R₃ is CN or C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, from a racemate of formula (V) and converting the substantially enantiomerically pure compound of formula (V) into a substantially enantiomerically pure compound of formula (IV).

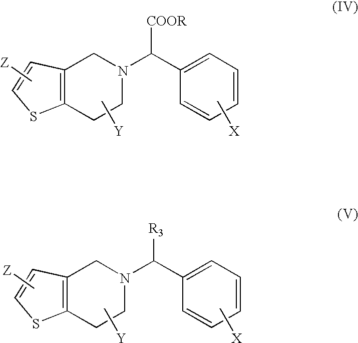

### DESCRIPTION

The present invention relates to a process for preparing enantiomerically pure α-phenyl-α-(6,7-dihydro-4H-thieno[3,2-c]pyridin-5-yl)-acetic acid derivatives and to certain novel enantiomerically pure α-phenyl-α-(6,7-dihydro-4H-thieno[3,2-c]pyridin-5-yl)-acetonitriles and acetamides.

Many of the early methods proposed for preparing enantiomerically pure α-phenyl-α-(6,7-dihydro-4H-thieno[3,2-c]pyridin-5-yl)-acetic acid derivatives, such as the platelet aggregation inhibitor Clopidogrel (1) and its analogues, involved the separation of the desired enantiomeically pure compound from a racemic α-phenyl-α-(6,7-dihydro-4H-thieno[3,2-c]pyridin-5-yl)-acetic acid derivative prepared by reacting a 4,5,6,7-tetrahydrothieno[3,2-c]pyridine with an α-halo-acetic acid derivative. See, for example, U.S. Pat. No. 4,529,596, U.S. Pat. No. 5,189,170 and U.S. Pat. No. 4,847,265.

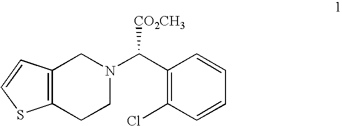

Both steps of these known syntheses, however, have significant drawbacks when practiced on an industrial scale. For example, the α-halo-acetic acid derivatives employed in the first step are lachrymators and irritants and the final resolution step is problematic for certain of the acid and ester derivatives, as the compounds concerned are oily and, thus, difficult to isolate.

Alternative processes, which do not involve the use of α-halo-acetic acid derivatives, but leave the formation of the hydropyridinyl ring until a final stage, have subsequently been proposed. See, for example, the processes disclosed in WO 98/51689, WO 98/51682, WO 98/51681, U.S. Pat. No. 5,204,469 and U.S. Pat. No. 6,080,875. It has been suggested that a further advantage of these processes is that they can be worked in a stereospecific manner, such that the configuration of an optically pure starting material can be preserved in the end product and a final resolution step is thus not required. See, for example, the process described in U.S. Pat. No. 6,080,875. However, all of these processes are longer and less convergent than the earlier processes using α-halo-acetic acid derivatives.

Accordingly, in a first aspect, the present invention provides a process for preparing a substantially enantiomerically pure compound of formula IV, or a pharmaceutically acceptable salt thereof:— 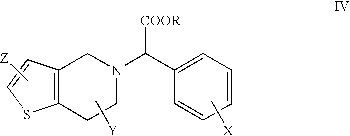
wherein R is hydrogen or a C₁-C₄ alkyl group and X, Y and Z are any atom or group, comprising a step of isolating a substantially enantiomerically pure compound of formula V:— 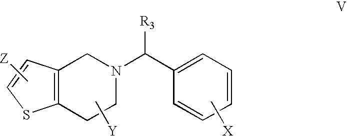
wherein R₃ is CN or C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, from a racemate of formula V and converting the substantially enantiomerically pure compound of formula V into a substantially enantiomerically pure compound of formula IV.

Racemic compounds of formula V can be produced without using an α-halo-acetic acid derivative and the inventors have determined that they can easily be resolved into enantiomers. Furthermore, once resolved, enantiomenrically pure compounds of formula V can be converted into enantiomenrically pure compounds of formula IV with ease and without any significant loss of enantiomeric purity. Therefore, by eliminating the need to carry out the difficult final resolution step or use the unpleasant starting materials employed in the aforementioned earlier known processes, without involving the degree of complexity involved in their proposed replacements, in which the hydropyridinyl ring is formed in a final step, the present invention allows a majority, if not all of the above discussed disadvantages of previously proposed process for preparing compounds of formula IV to be avoided.

A further advantage of processes in accordance with the invention, is that they allow enantiomenrically pure compounds of formula IV to be prepared in high yields and for any unwanted enantiomer to be racemised and subjected to a repeat of the inventive process.

In preferred embodiments of the invention, Y and Z are each independently hydrogen or a C₁-C₄ alkyl group. Preferably, both Y and Z are hydrogen. X is preferably a halogen and more preferably chlorine. In further preferred embodiments, X is bound to the carbon atom in the 2 position in the phenyl group in formulae IV and V.

R is preferably a C₁-C₄ alkyl group and most preferably a methyl group. R₃ is preferably C(O)NR₁R₂, with R₁ and R₂ being as defined above and, preferably, hydrogen. When R₁ and R₂ form a ring, it can be a cycloalkyl or a cycloalkenyl group that includes the amido nitrogen. The ring can include a further hetero-atom and can carry one or more substituent groups. The ring is preferably unsubstituted.

In a particularly preferred embodiment of the first aspect of the invention, R₃ is C(O)NR₁R₂, R₁ and R₂ are as-previously defined, and the racemate of formula V is prepared in a preliminary step by subjecting a racemic compound of formula V, wherein R₃ is CN, to hydrolysis, preferably under basic conditions. This preliminary step is preferably carried out by employing an alkali metal carbonate and hydrogen peroxide in a suitable, preferably protic, solvent. The preferred alkali metal carbonate is potassium carbonate and the preferred solvent includes a lower C₁-C₄ alkyl alcohol and is preferably a mixture of methanol and dimethylsulphoxide (DMSO).

In further preferred embodiments of the first aspect of the invention, racemic compounds of formula V, wherein R₃ is CN, are prepared by reacting a 4,5,6,7-tetrahydro[3,2-c]thienopyridine of formula VI:— 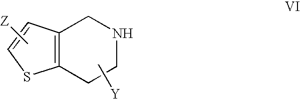
wherein Y and Z are as previously defined, or a salt thereof, with a benzaldehyde of formula VII:— 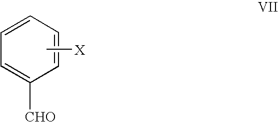
wherein X is as previously defined, or a derivative thereof, in the presence of a nitrile.

Preferably, the nitrile is in the form of an alkali metal cyanide salt and it is preferred for this reaction to be carried out in a protic solvent or mixture of protic solvents. Preferred such solvents include water and lower C₁-C₄ alkyl alcohols and preferred such mixed solvents include mixtures of water and lower C₁-C₄ alkyl alcohols. It is further preferred for this reaction to be carried out in the absence of any added acid and for the alkali metal cyanide salt to be combined (in any order) directly with the compounds of formulae VI and VII. In this last regard, although it is possible to carry out this reaction using a derivative of the benzaldehyde of formula VII, such as a bisulphite addition product thereof, it is preferred to avoid the use of such compounds or intermediates.

The step of isolating or resolving a substantially enantiomerically pure compound of formula V from a racemate of formula V preferably involves the formation of a salt of the racemate with an optically active acid, the isolation of a substantially optically pure form of this salt that includes the desired enantiomer of formula V, but substantially none of its mirror image (i.e. a substantially pure single stereoisomer of the salt), followed by the liberation of the desired enantiomer of formula V in a substantially pure form, for example, by the addition of a base.

The stereoisomer containing the desired enantiomer of formula V can be isolated in a substantially optically pure form by repeated recrystallisation from a solution of the racemic salt in a suitable solvent, for example, in the manner described in U.S. Pat. No. 4,847,265.

Alternatively, and in accordance with a second aspect of the present invention, a solution of a salt of the racemate of formula V with a single enantiomer of an optically active acid can be acidified sufficiently to cause a single stereoisomer of the salt to precipitate in a substantially pure form (i.e. in substantial isolation from the other stereoisomer). By acidified, it is meant that the solution should be rendered more acidic, but not necessarily acidic in absolute terms (although this is possible). Preferably, the enantiomer of the optically active acid used to form the salt is chosen so that the stereoisomer caused to precipitate is that which includes the desired enantiomer of formula V. The preferred desired enantiomers of formula IV and V are the dextro-rotatory (+) or S enantiomers. Acidification is preferably achieved by the expedient of adding an acid to the solution and the preferred acids used for this purpose are carboxylic acids, preferably the lower C₁-C₄ alkyl carboxylic acids and most preferably formic acid. Suitable solvents for use in this step include lower C₁-C₄ alkyl alcohols and ketones, the preferred solvents being methanol and acetone, preferably in addmixture.

The optically active acid used in the practice of the present invention is preferably a substantially enantiomerically pure form of camphor-10-sulphonic acid. When it is desired to isolate the dextro-rotatory (+) or S enantiomer of a compound of formula V, it is preferred to employ the (1S)-(+)-enantiomer of camphor-10-sulphonic acid, as it is the stereoisomers which include the desired dextro-rotatory (+) or S enantiomers of its salts with compounds of formula V that precipitate from solution on treatment with an acid in the aforementioned manner.

Thus, in a further aspect, the invention provides a process for preparing a substantially enantiomerically pure compound of formula V, or a pharmaceutically acceptable salt thereof:— 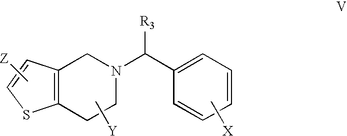
wherein R₃ is CN or C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, from a racemate of formula V, comprising forming a salt of the racemate with a single enantiomer of an optically active acid and isolating a substantially pure single stereoisomer thereof that includes the desired enantiomer of formula V.

After the precipitate of the stereoisomer that includes the desired enantiomer of formula V has been removed, for example by filtration, the mother liquor can be subjected to epimerisation, for example by the addition of a strong base, and the salt formation and resolution procedure repeated. The whole sequence of salt formation, resolution and epimerisation can be repeated as often as is necessary and practical in order to increase the overall yield of the final enantiomerically pure product.

As noted above, the desired enantiomer of formula V can be liberated from the isolated salt by the addition of a base. The preferred base for this purpose is an alkali metal bicarbonate, preferably sodium bicarbonate, and the liberation reaction is preferably carried out by adding a solution of the latter to a solution of the resolved salt in a mixture of a lower C₁-C₄ alkyl alcohol, preferably methanol, and water, to precipitate the desired enantiomer of formula V.

In preferred embodiments of the invention, substantially enantiomerically pure compounds of formula V are converted into substantially enantiomerically pure compounds of formula IV by one or a combination of the following techniques. When R₃, in the substantially enantiomerically pure compound of formula V, is CN, the compound is firstly converted into an equivalent substantially enantiomerically pure compound wherein R₃ is C(O)NR₁R₂ and R₁ and R₂ are as previously defined, by a method of the nature described above for the preparation of racemic compounds of formula V wherein R₃ is C(O)NR₁R₂. Substantially enantiomerically pure compounds of formula V, wherein R₃ is C(O)NR₁R₂ and R₁ and R₂ are as previously defined, can be converted, in accordance with the invention, into the corresponding substantially enantiomerically pure compounds of formula IV by acid catalysed hydrolysis, when R is hydrogen, or acid catalysed alkanolysis when R is a C₁-C₄ alkyl group. Preferably the alkanolysis is carried out using an acid, preferably sulphuric acid, and the corresponding C₁-C₄ alcohol. Thus, in a preferred embodiment, wherein R is methyl, this step involves the treatment of a substantially enantiomerically pure compound of formula V, wherein R₃ is C(O)NR₁R₂, with sulphuric acid in methanol. In preferred embodiments, this latter reaction is carried out in the presence of dimethylsulphate.

If desired, the substantially enantiomerically pure compounds of formula V can be converted into pharmaceutically acceptable acid addition salts using conventional techniques. The preferred such salt is the sulphuric acid salt.

Certain of the intermediates prepared in the practice of processes in accordance with the first aspect of the invention are novel and are the subjects of further aspects of the invention. These include the substantially enantiomerically pure amides of general formulae II and III, the substantially enantiomerically pure nitriles of general formulae IIA and IIIA and their substantially enantiomerically pure salts, wherein R₁, R₂, X, Y and Z are as defined above;

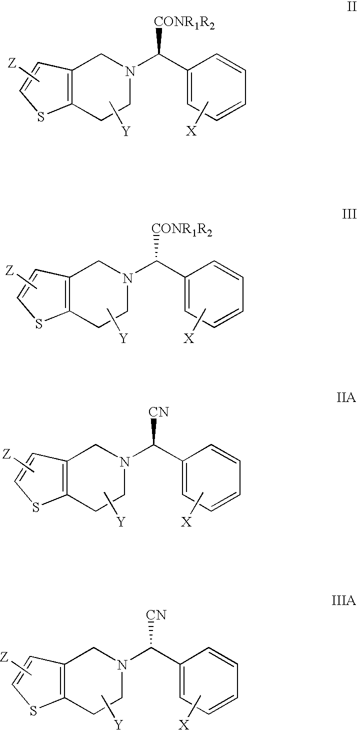

Preferred embodiments of these further aspects of the invention are (+)-α-(2-Chlorophenyl)-α-(6,7-dihydro-4H-thieno[3,2-c]pyridin-5-yl) acetamide and (+)-α-(2-Chlorophenyl)-α-(6,7-dihydro-4H-thieno [3,2-c]pyridin-5-yl) acetonitrile.

The preferred substantially enantiomerically pure compounds of formula IV prepared by processes in accordance with the present invention ate the methyl-α-(2-halophenyl)-α-(6,7-dihydro-4H-thieno[3,2-c]pyrid-5-yl)acetates, preferably the (+)-methyl-α-(2-halophenyl)-α-(6,7-dihydro-4H-thieno[3,2-c]pyrid-5-yl) acetates. The most preferred such compound is (+)-methyl-α-(2-chlorophenyl)-α-(6,7-dihydro-4H-thieno[3,2-c]pyrid-5-yl) acetate (Clopidogrel).

Where a compound is referred to as being substantially enantiomerically pure, or as being a substantially pure single stereoisomer, it will include less than 50, 20, 15, 10, 5, 2, 1, 0.5 or 0.1% w/w of any other enantiomer or stereoisomer of the same compound.

Compounds of formula II, IIA, Ill, IIIA, IV, and V can be in the form of acid addition salts, such as those formed by the addition of hydrochloric or sulphuric acid to the parent compound.

The preparation of (+)-(S)-methyl-α-(2-chlorophenyl)-m-(6,7-dihydro-4H-thieno [3,2-c]pyrid-5-yl)acetate (clopidogrel (1)) outlined in Scheme One is an example of a procedure comprising the process of the current invention. Compounds of the current invention are also exemplified in Scheme One and the process illustrated in this scheme is described in the examples which follow it.

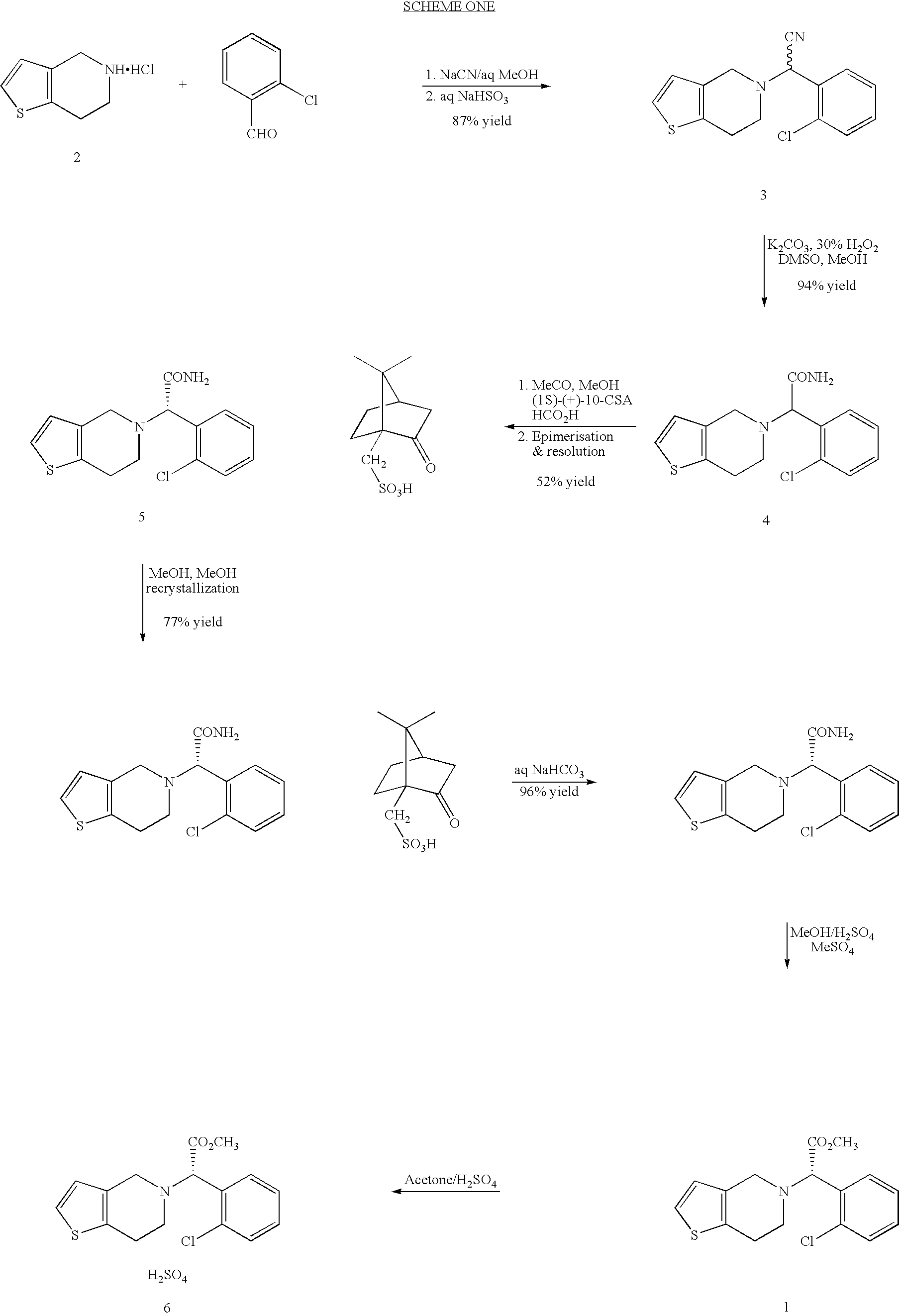

### EXAMPLE 1

Preparation of 2-Chlorophenyl-(6,7-dihydro-4H-thieno[3,2-c]pyridin-5-yl) acetonitrile (3)

To a mixture of Methanol (2.50 litres) and water (250.0 ml) was charged 4,5,6,7-tetrahydro[3,2-c]thienopyridine hydrochloride (2,500 g; 2.847 moles) with stirring. After stirring for 30 min, Sodium cyanide (153.0 g; 3.12 mol) was added and stirred further for 30 min. 2-Chlorobenzaldehyde (392.14 g; 2.79 mol) was added slowly to this reaction mixture between 23 and 28° C. over a period of 1.5 hours. This addition was exothermic and the temperature rose by 10° C. at the end of the addition. After the addition was over, the flask was heated in an oil bath between 40 and 50° C. and maintained at this temperature for 3 hours. After cooling the reaction mixture to 25-30° C., 5% sodium metabisuphite solution (250 ml) was added and stirred for 60 min at this temperature range. To the resulting slurry, water (7.5 litres) was added and stirred for 1.0 hour at 25-30° C. The off-white solid thus formed was filtered, washed with 1:1 mixture of methanol:water (2.5 litres) and the wet cake was dried at 75° C. under vacuum for 10 hours to obtain the product (3) as an an off-white solid. Weight: 720.0 grams (87.6%) mp: 121-123.6° C.

ESI-MS: 289.3 (M+H)+

Elemental analysis: Found: C: 62.74%; H: 4.54%; N: 10.03%

Calcd. for C₁₅H₁₃N₂SCl: C 62.37%; H 4.53%; N 9.69%

¹³C NMR (CDCl₃,): 135.4, 133.7, 133.3, 131.7, 131.2, 130.8, 127.6, 125.8, 123.8, 115.9, 59.9, 50.2, 48.5, 26.3

¹H NMR (CDCl₃,): 7.69 (m, 1H), 7.44 (m, 1H), 7.37 (m, 2H), 7.08 (d, 5.1 Hz, 1H), 6.70 (d, 5.1 Hz, 1H), 5.30 (s, 1H), 3.72 (AB quartet, 2H), 2.97 (m, 2H), 2.85 (m, 2H)

### Example 2

Preparation of 2-Chlorophenyl-(6,7-dihydro-4H-thieno[3,2-c]pyridin-5-yl)acetamide (4)

Charged 2-Chlorophenyl-(6,7-dihydro-4H-thieno[3,2-c]pyridin-5-yl)acetonirile (3, 706 g; 2.445 moles) to methanol (3.53 litres) at 23-28° C. with stirring. To this slurry, potassium carbonate (169 g; 1.222 moles) was added followed by dimethyl sulfoxide (261 ml; 3.667 moles). The contents were heated between 30 and 40° C. and 30.0% aqueous hydrogen peroxide solution (378 ml; 3.668 moles) was added between 40 and 50° C. slowly over a period of 4.0 hours. After the addition was over, the reaction mixture was maintained at this temperature further for an hour after which the reaction was brought to 20 to 30° C. 35% Hydrochloric acid (212.0 ml) in water (10.6 litres) was added slowly to the reaction mixture over a period of 1 hour. After stirring for an hour, the solid formed was filtered and washed with 1:1 methanol:water mixture (3.53 litres). The isolated solid (4) was dried in vacuum between 70-75° C. for a period of 12 hours. Yield=694 g (91.2%). mp: 124-127° C.

ESI-MS: 307.2 (M+H)⁺

Elemental analysis: Found: C: 58.85%; H: 4.94%; N: 9.40%

Calcd. for C₁₅H₁₅N₂OSCl: C 58.71%; H 4.92%; N 9.12%

¹³C NMR (CDCl₃,): 174.4, 136.1, 134.2, 134.0, 133.8, 131.1, 130.7, 130.1, 127.7, 125.9, 123.7, 70.0, 51.6, 49.9, 26.6

¹H NMR (CDCl₃,): 7.50 (m, 1H), 7.44 (m, 1H), 7.28 (m, 2H), 7.15 (bs, 1H, D₂O exchangeable), 7.08 (d, 5.1 Hz, 1H), 6.67 (d, 5.1 Hz, 1H), 5.77 (bs, 1H, D₂O exchangeable), 4.91 (s, 1H), 3.62 (AB quartet, 2H), 2.90 (bs, 4H)

### Example 3

Preparation of (+)-Camphor-10-sulfonic acid salt of (2-Chlorophenyl) (6,7-dihydro-4H-thieno[3,2-c]pyridin-5-yl)acetamide (5)

To a stirred slurry of (2-Chlorophenyl) (6,7-dihydro-4H-thieno[3,2-c]pyridin-5-yl) acetamide (2.32 moles) in acetone (3.56 litres) and methanol (0.356 litres) maintained at 23-28° C. was added a solution of (1S)-(+)-Camphor-10-Sulfonic acid (2690.87 g; 1.162 moles) dissolved in acetone (1.43 litres) over a period of 1-1.5 hour. After stirring for another hour, formic acid (53.47 g; 1.162 mol) was added all at once and stirred for 1.0 hour after which the reaction mixture was cooled to 5-15° C. and kept at this temperature for another 1.0 hour. The solid thus formed was filtered and washed with acetone (1.78 litres) and dried in vacuum oven between 60 and 65° C. for a period of 5 hours. Yield: 420.0 g (33.5% by theory based on the enantiomer content).

MP: 203.1-205° C.; α_{D}²⁵ =+46.4 (c=1.0 g/100 ml; methanol)

The (+)-Camphor-10-sulfonic acid salt of (2-Chlorophenyl) (6,7-dihydro-4H-thieno[3,2-c]pyridin-5-yl)acetamide (428.0 g; 1.17 moles) obtained was charged into methanol (1.57 litres) with stirring at 23 to 28° C. The contents were heated between 50 to 60° C. and the temperature was maintained at this temperature for 2.0 hours. To this clear solution, acetone (6.28 litres) was added and the temperature was maintained at this temperature for 1 hour. The reaction was cooled between 5-15° C. and stirred for another hour. The solid thus precipitated was filtered and dried under vacuum for 4 hours.

Yield: 323.6 g (75.6% by theory). Mp: 215-221° C.; α_{D}²⁵=+50.9 (c=1.0 g/100 ml; methanol).

ESI-MS: 307.2 (M+H)⁺

Elemental analysis: Found: C 55.51%; H 5.82%; N 5.54%

Calcd. for C₂₅H₃₁N₂O₅S₂Cl: C 55.69%; H 5.79%; N 5.19%

¹³C NMR (CDCl₃,): 215.6, 166.9, 134.2, 131.4, 131.3, 129.9, 129.8, 128.5, 127.9, 127.7, 125.1, 124.6, 65.3, 58.0, 49.8, 48.5, 46.6, 42.0, 26.1, 24.0, 21.7, 19.8, 19.2

¹H NMR (CDCl₃): 7.80 (d, 7.4 Hz, 1H), 7.61 (d, 7.7 Hz, 1H), 7.49 (m, 2H,) 7.38 (d, 5.2 Hz, 1h), 6.82 (d, 5.2 Hz, 1H), 5.35 (s, 1H), 4.2-3.9 (m, 2H), 3.45 (m, 2H), 3.09 (m, 2H) multiple signals between 3.0-0.7

### Example 4

Preparation of (+)-(2-Chlorophenyl) (6,7-dihydro-4H-thieno[3,2-c]pyridin-5-yl) acetamide [(+)-(4)]

The crystallized (+)-Camphor-10-sulfonic acid salt of (2-Chlorophenyl) (6,7-dihydro-4H-thieno[3,2-c]pyridin-5-yl)acetamide (5, 470.0 g; 0.872 moles) was charged into methanol (2.35 litres) with stirring at 23 to 28° C. followed by water (0.94 litres). To this clear solution, activated carbon (9.4 g) was added and the contents were stirred for 1.5 hours at this temperature. The activated carbon was filtered-off by passing the contents of the flask through a bed of celite on a Buchner funnel and the residue in the funnel was washed with Water: Methanol mixture (2:5 ratio; 0.47 litre). To the combined filtrate, 2% (w/v) aqueous sodium bicarbonate solution (3.76 litres) was added over a period of 30 minutes and stirred for 1.0 hour.

The solid precipitated was filtered, washed with methanol: water (1.88 litres: 1:1 v/v) and dried under vacuum for a period of 8 hours between 70 and 75° C.

Yield: 258.0 g (96.5% by theory). Mp: 150-153° C.; α_{D}²⁵ =+40.2 (c=1.0 g/100 ml; methanol).

ESI-MS: 307.2 (M+H)⁺

Elemental analysis: Found: C 56.59%; H 4.80%; N 9.08%

Calcd. for C₁₅H₁₅N₂OSCl: C 58.71%; H 4.92%; N 9.12%

¹³C NMR (CDCl₃,): 174.3, 136.2, 134.3, 133.9, 131.2, 130.8, 130.1, 127.7, 125.9, 123.7, 70.1, 51.7, 49.9, 26.6

¹H NMR (CDCl₃,): 7.50 (m, 1H), 7.44 (m, 1H), 7.28 (m, 2H), 7.14 (bs, 1H, D₂O exchangeable), 7.08 (d, 5.1 Hz, 1H), 6.67 (d, 5.1 Hz, 1H), 5.77 (bs, 1H, D₂O exchangeable), 4.91 (s, 1H), 3.62 (AB quartet, 2H), 2.90 (bs, 4H)

### Example 5

Preparation of (+)-(S)-(2-Chlorophenyl) (6,7-dihydro-4H-thieno[3,2-c]pyridin-5-yl)acetic acid methyl ester (1)

Concentrated sulfuric acid (98%; 31.3 ml; 0.587 moles) was charged in to methanol (105 ml) with stirring between 0 and 10° C. followed by dimethyl sulfate (15 ml; 0.157 mol). The contents were heated to reflux for 1.5 hours after which the reaction mixture was cooled to 25-30° C. and (+)-(2-Chlorophenyl) (6,7-dihydro-4H-thieno[3,2-c]pyridin-5-yl)acetamide [(+)-4, 30.0 g; 97.8 m.moles] was charged. The reaction mixture was heated and maintained between 75 and 85° C. for a period of 35 hours. The reaction mixture was cooled to 25-30° C. and pouted in to water (600 ml) with stirring. Dichloromethane (300 ml) was added, stirred for 1 hour after which the organic layer was separated. To the aqueous layer dichloromethane (150 ml) was added and stirred for 1 hour and the separated organic layer was combined with the earlier one and washed with water (150 ml). 5% (w/v) aqueous sodium bicarbonate (150 ml) solution was added to this organic layer and stirred for a period of an hour and the separated organic layer was washed with water (150 ml) and treated with activated carbon (2.4 g) for a period of 3 hours with stirring. The activated carbon was removed by filtration through celite bed and the celite bed was washed with Dichloromethane (30 ml). This washing was coupled with the filtrate and the solvent removed under vacuum to yield (+)-(2-Chlorophenyl) (6,7-dihydro-4H-thieno[3,2-c]pyridin-5-yl)acetic acid methyl ester (1) as a pale yellow oil.

Yield: 22.0 g (69.9% by theory).

### EXAMPLE 6

Preparation of (+)-(S)-(2-Chlorophenyl) (6,7-dihydro-4H-thieno[3,2-c]pyridin-5-yl)acetic acid methyl ester. H₂SO₄ salt (6)

To a stirred solution of (+)-(S)-(2-Chlorophenyl) (6,7-dihydro-4H-thieno [3,2-c]pyridin-5-yl)acetic acid methyl ester (1, 22 g; 68.4 m.moles) in acetone (132 ml) was added 98% sulfuric acid (0.36 ml; 6.84 m.moles) with stirring between 20-28° C. The contents were stirred for 5 hours. The reaction mixture was cooled between 0 and 10° C. and the temperature was maintained at this temperature for 2.0 hours and the precipitated solid was removed by filtration. To the filtrate, 98% sulphuric acid (3.28 ml; 0.0615 moles) in ethyl acetate (44 ml) was added over a period of an hour between 20 and 28° C. After stirring for 5 hours, the precipitated solid was filtered, washed with acetone (66 ml) and dried in vacuum oven between 50 and 55° C. for 4 hours.

Yield: 16.5 g (57.5% by theory). Mp: 186.7-187.4° C.; α_{D}²⁵=+54.5 (c=1.89 g/100 ml; methanol)

ESI-MS: 322.1 (M+H)⁺

Elemental analysis: Found: C: 46.08%; H: 4.35%; N: 3.64%

Calcd. for C₁₆H₁₈NO₆S₂C1: C: 45.76%; H: 4.32%; N: 3.33%

¹³C NMR (CDCl₃,): 167.8, 133.9, 131.6, 131.2, 130.0, 127.8, 125.1, 124.2, 65.8, 52.8, 50.0, 48.5, 22.7

¹H NMR (CDCl₃,): 7.63-7.45 (m, 4H), 7.35 (d, 5.1 Hz, 1H), 6.79 (d, 5.1 Hz, 1H), 5.54 (s, 1H), 4.21-3.9 (m, 2H), 3.71 (s, 3H), 3.45 (m, 2H), 3.04 (broad triplet, 2H)

1. A process for preparing a substantially enantiomerically pure compound of formula IV, or a pharmaceutically acceptable salt thereof:— 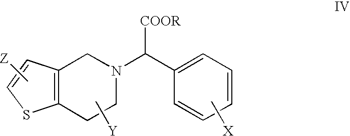
wherein R is hydrogen or a C₁-C₄ alkyl group and X, Y and Z are any atom or group, comprising a step of isolating a substantially enantiomerically pure compound of formula V:— 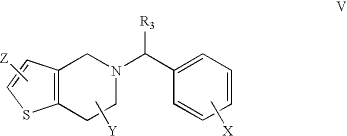
wherein R₃ is CN or C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, from a racemate of formula V and converting the substantially enantiomerically pure compound of formula V into a substantially enantiomerically pure compound of formula IV.

2. The process of claim 1, wherein R is a C₁-C₄, alkyl group.

3. The process of claim 1 wherein R₃ is C(O)NR₁R₂.

4. The process of claim 1 wherein R₁ and R₂ are hydrogen.

5. The process of claim 1 wherein Y and Z are each independently hydrogen or a C₁-C₄ alkyl group.

6. The process of claim 1 wherein both Y and Z are hydrogen.

7. The process of claim 1 wherein X is a halogen.

8. The process of claim 1 wherein X is bound to the carbon atom in the 2 position in the phenyl group in formulae IV and V.

9. The process of claim 1 wherein R₃ is C(O)NR₁R₂ and the racemate of formula V is prepared in a preliminary step comprising subjecting a racemic compound of formula V, wherein R₃ is CN, to hydrolysis.

10. The process of claim 9 wherein said preliminary step is carried out by employing an alkali metal carbonate and hydrogen peroxide in a protic solvent.

11. The process of claim 1 wherein the racemate or racemic compound of formula V wherein R₃ is CN is prepared by reacting a 4,5, 6,7-tetrahydro[3,2-c]thienopyridine of formula VI:— 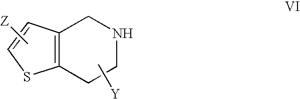
wherein Y and Z are any atom or group or a salt thereof, with a benzaldehyde of formula VII:— 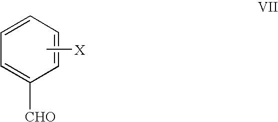
wherein X is any atom or group, or a derivative thereof, in the presence of a nitrile.

12. The process of claim 11 wherein the nitrile is in the form of an alkali metal cyanide salt and the compounds of formulae VI and VII are reacted in a protic solvent or mixture of protic solvents.

13. The process of claim 11 wherein the compounds of formulae VI and VII are reacted in the absence of any added acid and the alkali metal cyanide salt is combined directly with the compounds of formulae VI and VII.

14. The process of claim 1 wherein the step of separating the substantially enantiomerically pure compound of formula V from a racemate of formula V comprises the steps of forming a salt of the racemate with an optically active acid and isolating the substantially optically pure form of this salt that includes the desired enantiomer of formula V.

15. The process of claim 14, wherein a solution of a salt of the racemate of formula V with a single enantiomer of an optically active acid is acidified sufficiently to cause a single stereoisomer of the salt to precipitate in a substantially pure form.

16. A process for preparing a substantially enantiomerically pure compound of formula V, or a pharmaceutically acceptable salt thereof:— 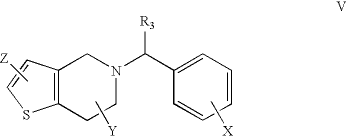
wherein R₃ is CN or C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, from a racemate of formula V, comprising forming a salt of the racemate with a single enantiomer of an optically active acid and isolating a substantially pure single stereoisomer thereof that includes the desired enantiomer of formula V.

17. The process of claim 16, wherein a solution of a salt of the racemate of formula V with a single enantiomer of an optically active acid is acidified sufficiently to cause the single stereoisomer of the salt to precipitate in a substantially pure form.

18. The process of claim 15 or claim 17, wherein sufficient acid is added to cause the desired stereoisomer to precipitate from a solution of the salt of the racemate of formula V.

19. The process of claim 18, wherein the acid is a lower C₁-C₄ alkyl carboxylic acid, preferably formic acid.

20. The process of claim 15 or claim 17 wherein the solvent is a lower C₁-C₄ alkyl alcohol, a ketone, or a mixture of such an alcohol and ketone.

21. The process of claim 14 or claim 16 wherein the optically active acid is a substantially enantiomerically pure form of camphor-10-sulphonic acid.

22. The process of claim 15 or claim 16 wherein the substantially enantiomerically pure compound of formula V is a substantially pure dextro-rotatory (+) or S enantiomer and the optically active acid is (1S)-(+)-camphor-10-sulphonic acid.

23. The process of claim 15 or claim 16 wherein the substantially enantiomerically pure compound of formula V is liberated from the isolated salt by the action of a base.

24. The process of claim 1 or claim 16 wherein a substantially enantiomerically pure compound of formula V, wherein R₃ is C(O)NR₁R₂ and R₁ and R₂ are each independently hydrogen or a C₁-C₄ alkyl group, or, together with the nitrogen in the C(O)NR₁R₂ group, form a ring that includes 2-6 carbon atoms, is converted into the corresponding substantially enantiomerically pure compound of formula IV by acid catalysed hydrolysis, when R is hydrogen, or acid catalysed alkanolysis when R is a C₁-C₄ alkyl group.

25. The process of claim 24, wherein the alkanolysis is carried out using sulphuric acid, and the corresponding C₁-C₄ alcohol.

26. The process of claim 1 or claim 16 wherein the substantially enantiomerically pure compounds of formula VI and V are dextrorotatory (+) or S enantiomers.

27. A substantially enantiomerically pure amide of formula II or III, wherein R₁ and R₂ are each independently hydrogen or a C₁-C₄ alkyl group, or, together with the nitrogen in the C(O)NR₁R₂ group, form a ring that includes 2-6 carbon atoms, and X, Y and Z are any atom ore group:— 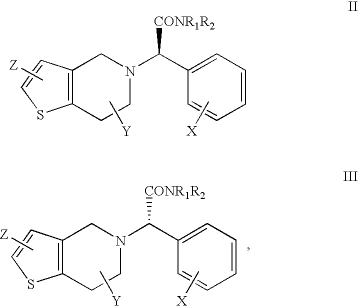
or a salt thereof

28. A substantially enantiomerically pure nitrile of general formula IIA or IIIA wherein R₁ and R₂ are each independently hydrogen or a C₁-C₄ alkyl group, or, together with the nitrogen in the C(O)NR₁R₂ group, form a ring that includes 2-6 carbon atoms and X, Y and Z are any atom ore group:— 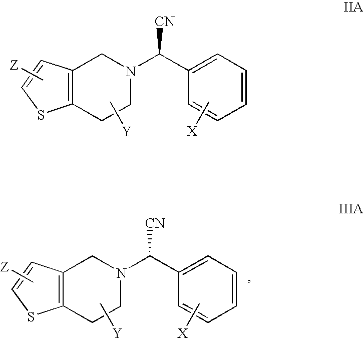
or a salt thereof

29. (+)-α-(2-halophenyl)-α-(6,7-dihydro-4H-thieno[3,2-c]pyridin-5-yl) acetamide, or a salt thereof.

30. (+)-α-(2-halophenyl)-α-(6,7-dihydro-4H-thieno[3,2-c]pyridin-5-yl) acetonitrile, or a salt thereof.

31. (+)-α-(2-chlorophenyl)-α-(6,7-dihydro-4H-thieno[3,2-c]pyridin-5-yl) acetamide, or a salt thereof.

32. (+)-α-(2-chlorophenyl)-α-(6,7-dihydro-4H-thieno[3,2-c]pyridin-5-yl) acetonitrile, or a salt thereof.

33. (canceled)

34. A substantially enantiomerically pure compound of formula IV or V prepared by the process of claim 1 or claim 16 or a part of such a process.

35. A (+)-methyl-α-(2-halophenyl)-α-(6,7-dihydro-4H-thieno[3,2-c]pyrid-5-yl) acetate prepared by the process of claim 1 or claim 16.

36. Methyl-α-(2-chlorophenyl)-α-(6,7-dihydro-4H-thieno[3,2-c]pyrid-5-yl) acetate prepared by the process of claim 1 or claim 16.
